(19) United States
(12) Plant Patent Application Publication
(10) Pub. No.: US 2017/0238453 P1
(43) Pub. Date: Aug. 17, 2017

(54) Senecio plant named 'Sennova 0012015'

(50) ;

(71) Applicant: Floricultura Novazel S.A., Santiago (CL)

(72) Inventor: Matias Eduardo Avendano Ceballos, Santiago (CL)

(21) Appl. No.: 14/998,799

(22) Filed: Feb. 17, 2016

(51) Int. Cl. A01H 5/02; A01H 5/12

(52) U.S. Cl. PLT/480

(57) ABSTRACT

A new cultivar of Senecio plant, ‘Sennova 0012015’, characterized by its leaves that are broadly ovate in shape, unlobed with wavy crenate margins, silvery white in color due to its densely woolly tomentose surface, its foliage that retains its coloration for a long period during the growing season, its ease of propagation, and its non-flowering habit.

Botanical classification: Senecio candicans.

Variety denomination: ‘Sennova 0012015’.

### BACKGROUND OF THE INVENTION

The present invention relates to a new and distinct cultivar of Senecio plant, botanically known as Senecio candicans ‘Sennova 0012015’ and will be referred to hereafter by its cultivar name, ‘Sennova 0012015’. The new cultivar represents a new dusty miller grown as an annual for bedding and container use.

The new cultivar resulted from on ongoing breeding program by the Inventor at his nursery in Puerto Montt, Chile with the objective of selecting a new cultivars of Senecio candicans with unique leaf shapes and colors and plant habits. ‘Sennova 0012015’ arose from open pollination of an unnamed and unpatented plant of Senecio candicans and therefore the male parent is unknown. The Inventor selected the new cultivar as a single unique plant from the resulting seedlings in the summer of 2010.

Asexual propagation of the new cultivar was first accomplished by division in Puerto Montt, Chile in May of 2011 by the Inventor. Asexual propagation by division, root cuttings and tissue culture has determined that the characteristics of this cultivar are stable and are reproduced true to type in successive generations.

### SUMMARY OF THE INVENTION

The following traits have been repeatedly observed and represent the characteristics of ‘Sennova 0012015’. These attributes in combination distinguish ‘Sennova 0012015’ as a new and distinct cultivar of Senecio.
1. ‘Sennova 0012015’ exhibits leaves that are broadly ovate and unlobed with wavy crenate margins.
2. ‘Sennova 0012015’ exhibits leaves that are silvery white in color due to its densely woolly tomentose surface.
3. ‘Sennova 0012015’ exhibits leaves that retains its coloration for a long period during the growing season.
4. ‘Sennova 0012015’ exhibits a moderately vigorous growth habit.
5. ‘Sennova 0012015’ has been shown to be readily propagated.
6. ‘Sennova 0012015’ has not been observed to flower.

The female parent of ‘Sennova 0012015’, differs from ‘Sennova 0012015’ in having a slower growth habit and in having leaves that are less white in color with poorer color retention during the growth season and that differs in shape. ‘Sennova 0012015’ can also be compared to cultivars of dusty miller; Brachyglottis greyi ‘Sunshine’ (not patented) and Senecio cineraria ‘Silver Dust’ (not patented). ‘Sunshine’ is similar to ‘Sennova 0012015’ in having foliage that is silver-grey in color. ‘Sunshine’ differs from ‘Sennova 0012015’ in having leaves that are much smaller in size, in having a woody shrub plant habit and in being very floriferous. ‘Silver Dust’ is similar to ‘Sennova 0012015’ in having foliage that is silver-grey in color. ‘Silver Dust’ differs from ‘Sennova 0012015’ in having leaves that are pinnately lobed.

### BRIEF DESCRIPTION OF THE DRAWINGS

The accompanying colored photographs illustrate the overall appearance and distinct characteristics of the new Senecio. The photographs were taken of a six month-old plant of ‘Sennova 0012015’ as grown in a cold greenhouse in a 3-liter container in Lisse, The Netherlands.

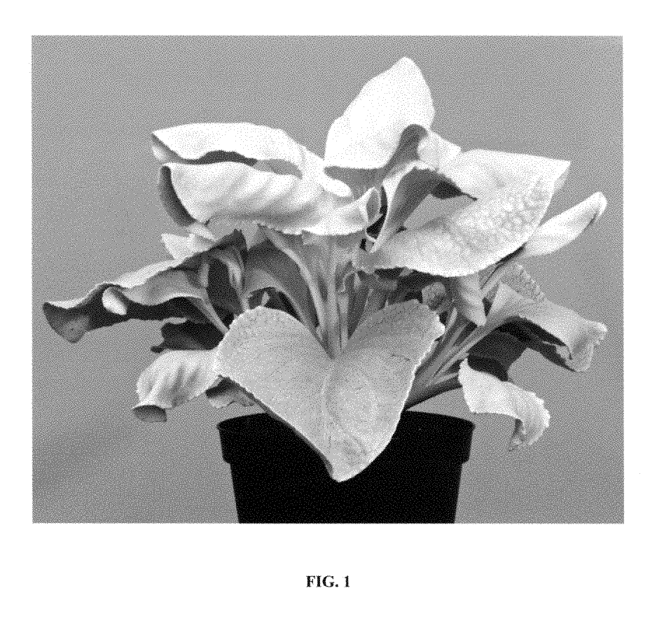

The photograph in FIG. 1 is a view of a plant of ‘Sennova 0012015’.

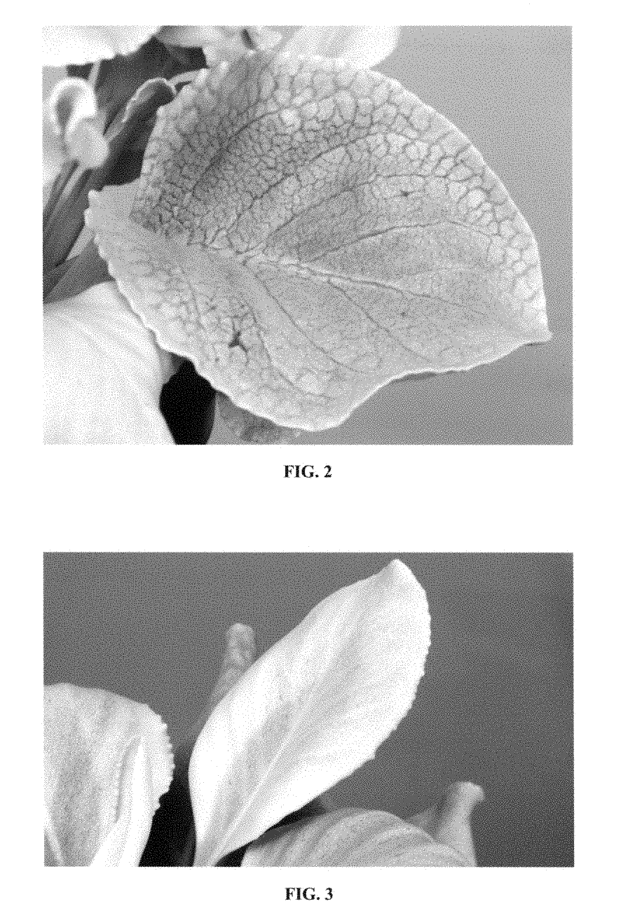

The photograph in FIG. 2 is a close-up view of a mature leaf of ‘Sennova 0012015’.

The photograph in FIG. 3 provides a close-up view of a young leaf of ‘Sennova 0012015’. The colors in the photographs may differ slightly from the color values cited in the detailed botanical description, which accurately describe the colors of the new Senecio.

### DETAILED BOTANICAL DESCRIPTION

The following is a detailed description of six month-old plants of the new cultivar as grown in 3-liter circular containers in a greenhouse in Lisse, The Netherlands. The phenotype of the new cultivar may vary with variations in environmental, climatic, and cultural conditions, as it has not been tested under all possible environmental conditions. The color determination is in accordance with the 2007 R.H.S. Colour Chart of The Royal Horticultural Society, London, England, except where general color terms of ordinary dictionary significance are used.
General description:
   Blooming period.—Has not been observed to bloom to date.
   Plant habit.—Herbaceous perennial, upright and broadly spreading.
   Height and spread.—An average of 50 cm in height and 60 cm in width as a six month-old plant.
   Cold hardiness.—At least to U.S.D.A. Zone 8.
   Diseases and pests.—No susceptibility or resistance to diseases or pests has been observed.
   Root description.—Fibrous.
   Propagation.—Division, root cuttings, and tissue culture.
   Growth rate.—Moderately vigorous.
   Root development.—6 to 8 weeks from a rooted cutting or a tissue culture plug.
Stem description:
   Stem color.—144A and covered with tomentose NN155C in color.
   Stem size.—Average of 6 cm in length and 9 mm in diameter.
   Stem surface.—Very densely wooly and tomentose NN155C in color.
   Branching habit.—Freely branching, strong, 1 basal branches, pinching not required, but will improve branching.
   Internode length.—Average of 1.1 cm.
Foliage description:
   Leaf division.—Simple.
   Leaf arrangement.—Alternate.
   Leaf shape.—Broadly ovate.
   Leaf size.—Average of 17.2 cm in length and 16 cm in width.
   Leaf number.—Average of 6 per lateral branch.
   Leaf base.—Broad attenuate.
   Leaf apex.—Rounded.
   Leaf margin.—Crennulate and wavy.
   Leaf venation.—Pinnate, upper surface color; 146D, densely covered with woolly tomentose 194D in color, lower surface color; vein surface color not visible due to densely woolly tomentose, N155A and NN155D in color.
   Leaf surface.—Upper surface has a slight sheen, both sides densely woolly tomentose N155A and NN155D in color.
   Leaf substance.—Leathery.
   Leaf color.—Leaf color is defined by the very dense woolly tomentose, young upper and lower surface; color between N155A and NN155D, mature upper surface; color between N155A and 198D, mature lower surface; color between N155A and NN155D.
   Petioles.—Sulcate in shape, average of 10.8 cm in length and 1.1 cm in diameter, upper surface; 146D in color with surface is densely covered with woolly tomentose 194D in color, lower surface; color defined by wooly tomentose surface N155A and NN155D in color.
   Foliage fragrance.—Mild mint scent if crushed.
Flower description: Flowering has not be observed to date.

It is claimed:

1. A new and distinct variety of Senecio plant designated ‘Sennova 0012015’ as described and illustrated herein.
